(19) United States
(12) Patent Application Publication
(10) Pub. No.: US 2006/0039960 A1
(43) Pub. Date: Feb. 23, 2006

(54) Plaster containing fentanyl

(76) Inventors: Günter Cordes, Leichlingen (DE); Ulrike Vollmer, Dormagen (DE)

Correspondence Address: FRIEDRICH KUEFFNER, 317 MADISON AVENUE, SUITE 910, NEW YORK, NY, 10017, US

(21) Appl. No.: 10/515,608

(22) PCT Filed: May 20, 2003
(86) PCT No.: PCT/DE03/01635

§ 371 Date: May 9, 2005

Related U.S. Application Data
(60) Provisional application No. 60/428,556, filed on Nov. 22, 2002.

(30) Foreign Application Priority Data
May 28, 2002 (DE) 102 23 835.9

(51) Int. Cl. A61K 9/70; A61K 31/445

(52) U.S. Cl. 424/449; 514/317

(57) ABSTRACT

The invention relates to a transdermal, therapeutic system comprising a coating layer and an adhesive matrix having a fentanyl content.

The invention concerns a transdermal therapeutic system with a cover layer, an adhesive matrix that contains fentanyl as the active ingredient, and a removable protective layer.

Fentanyl (fentanylum, fentanil) was already patented in 1984 for use in a transdermal patch (U.S. Pat. No. 4,588,580). In the interim it has proven extremely effective in the treatment of severe and/or chronic pain states, especially in the treatment of postoperative pain and pain associated with cancer. Side effects of fentanyl are the typical side effects observed with this class of substances, the opioids, namely, nausea, circulatory problems, constipation or pruritus, and life-threatening respiratory depression. This means that the substance must be supplied to the body slowly and continuously. Due to the poor bioavailability of <10%, oral sustained-release dosage forms (sustained-release tablets) cannot be used. When administered transdermally, the first-pass effect in the liver is avoided, the absorption of the substance through the skin is good, and long-lasting, uniform blood levels can be achieved in this way if a suitable transdermal formulation can be successfully developed. For these reasons, the administration of fentanyl from a transdermal patch is achieving a steadily increasing market share in the treatment of severe pain states.

In a transdermal system like Durogesic™, the fentanyl released from the formulation penetrates the skin barrier, enters the systemic circulation through the perfused subcutaneous tissue, and then develops its analgesic effect centrally by reaction at the opiate receptors in the brain. Of course, due to the highly lipophilic character of the opioid analogue, it becomes concentrated in the fatty tissue, from which it can later be released into the circulation; this is referred to as a skin depot.

The penetration of a drug through the skin is largely determined by the physicochemical properties of the substance. Mainly the octanol/water partition coefficient and the molecular size play a role here (R. O. Potts and R. H. Guy in: R. Gurny and A. Teubner: Dermal and Transdermal Drug Delivery, Wiss. Verlagsges., Stuttgart (1993)). Since the patient prefers to use an effective patch in a size that is as inconspicuous and small as possible, there is also the desire in this case to increase the penetration rate, for which there are actually only two possibilities if one does not wish to increase the skin by “microinjections”, microlesions, or the application of external energy sources (e.g., iontophoresis or the like):

1. Facilitation of diffusion by the addition of penetration accelerators or the use of electric voltage (iontophoresis).

2. Increasing the drug concentration in the base even beyond the solubility limit (supersaturation).

Substances used as penetration accelerators include alcohols, fatty acids, fatty alcohols, monohydric and polyhydric alcohols, laurocapram, and surfactants. However, many of these substances act by interfering with the barrier function of the skin and are thus more or less irritating to the skin. Nevertheless, numerous systems have been described in the patent literature (cf. WO 89/10,108, WO 99/56,782, WO 99/32,153, etc.).

Systems in which the active substance is present in supersaturated form are better tolerated. The maximum flux of a substance through the skin is usually limited by its solubility in the horny layer (stratum corneum), which constitutes the skin penetration barrier. This saturation concentration will become established if the active substance in the vehicle, e.g., in the matrix of the transdermal system, is also present in a concentration that corresponds to its solubility in the vehicle. One possible means of further increasing this so-called maximum thermodynamic activity consists in incorporating the drug in a concentration that exceeds the solubility in the vehicle. This is possible, for example, by incorporating the fentanyl in acrylate copolymers (WO 20024386). However, supersaturation must be established so sensitively that the supersaturation is as high as possible but as stable as necessary, since, as is well known, supersaturated systems are metastable and are converted to the saturated state by recrystallization after storage. This then has the disadvantage that, because of the crystallization, these systems lead to product complaints due to this deficient aspect and due to a lack of adhesiveness. Close contact between the transdermal system and the skin is likewise necessary to get an effective fraction of fentanyl into the target area of the blood circulation.

Of course, as has already been mentioned, fentanyl is among the few drugs which, due to its physicochemical properties, permeates the skin barrier very well and readily migrates into and accumulates in polymers. Since the therapeutic range of fentanyl is narrow and, in addition, there is the potential for addiction, as with all opioids, a further objective of the development of a transdermal fentanyl patch is to incorporate as little substance as possible but as much as necessary to be able to maintain a therapeutic blood level over a period of several days.

The objective of the present invention is to devise an improved transdermal therapeutic system of the type mentioned at the beginning.

In accordance with the invention, this objective is achieved by using an acrylate copolymer adhesive matrix that contains no penetration accelerators, such that the adhesive matrix is selected from the following group:
(a) basic acrylate copolymer, especially acrylate copolymer with hydroxyethyl acrylate units and with an organotitanium compound as a crosslinking agent; and
(b) basic acrylate copolymer with vinyl acetate units and free of crosslinking agents, especially acrylate copolymer with hydroxyethyl acrylate units and vinyl acetate units.

It was discovered that the incorporation of the fentanyl as a base into an acrylate copolymer crosslinked in a very specific way achieves saturation that is so stable that an effective product is obtained without the necessity of adding penetration accelerators and at the same time produces optimum adhesion to the skin of such a nature that during close contact between the dermal system and the outer skin barrier for several days up to a maximum of half a week, it can nevertheless be removed again at any time without producing either a sensation of pain or skin irritation.

Several acrylate copolymers produced by the company National Starch & Chemical, B.V., Zutphen, Netherlands (trade name: Durotak) were tested. It was found that a copolymer that contains small amounts of acrylic acid (Durotak 387-4350) and a graft copolymer (Durotak 87-9301 elite) that contains no acid or base groups but instead contains an acrylic octylamide graft are too reactive and lead to significant decomposition of fentanyl within a very short time. Adhesives without functional groups (Durotak 87-4098) were found to be sufficiently stable, but adhesives with a small proportion of hydroxyethyl acrylate (Durotak 387-2510) were clearly superior with respect to thermodynamic activity in the same concentration, which was apparent from better in vitro permeation rates in excised human skin mounted in Franz cells.

However, the use of an adhesive with hydroxyethyl acrylate (Durotak 387-2510) in the presence of fentanyl leads to softening of the polymer, which in turn leads to excessive adhesive strength and “cold flow” of the adhesive matrix. Both are undesirable and make a patch unsuitable.

Several types of solvent-based adjustment of the adhesive strength of this very specific acrylate copolymer were tested. The copolymers were produced by the company National Starch & Chemical, B.V., Zutphen, Netherlands under the trade name Durotak. The formulation compositions are reproduced in the following table (see next page):

| | | | | Example 1 in |
|---|---|---|---|---|
| | | | | Accordance |
| | Comparative | Comparative | Comparative | with the |
| Parameter | Example 1 | Example 2 | Example 3 | Invention |
| Durotak 387- | X | X | X | X |
| 2510 | | | | |
| crosslinking | — | 0.5% | 5% | 0.5% |
| agent | | aluminum | polybutyl | polybutyl |
| | | acetyl | titanate | titanate |
| | | acetonate | | |
| adhesive | 9.1 | 6.8 | 0.6 | 3.0 |
| strength in | | | | |
| vitro [N/25 | | | | |
| mm] | | | | |
| adhesive | painful | residues on | adheres too | good |
| properties | removal, | the skin | weakly | adhesive |
| in vivo | including | when patch | | property |
| | abrasion of | pulled off | | |
| | the horny | | | |
| | layer | | | |

As can be seen, the wearing properties are achieved by crosslinking the basic Durotak. There are many other possible means of influencing the cohesion and adhesive properties of these adhesives produced by National Starch & Chemical, B.V., Zutphen, Netherlands (Durotak 387-2510, 387-2516), e.g., by titanium crosslinking agents, or by the addition of solids, such as Aerosil or talc, which have been very successfully used in other systems (JP 2000-04447), or by the addition of other polymers, such as silicone, resins, or polyisobutylenes (WO 99/02141, WO 93/00058), but when only the aforesaid adhesive Durotak 387-2510 is used, the use of polybutyl titanate produces the best result, which was surprising. A specific, unknown type of incorporation of the active substance in the acrylate copolymer cavities, which are suitably adjusted by crosslinking, is apparently produced, without binding or irreversible inclusion occurring. This is also evident from the fact that when polybutyl titanate is added to a formulation with fentanyl, an adhesive strength in vitro of about 3 N/25 mm results, as listed in the table above, whereas the placebo, i.e., the formulation without fentanyl, has adhesive strength values that are higher by a factor of 2 (6 N/25 mm).

The incorporation of the titanium crosslinking agent requires certain skills on the part of the expert. Depending on the supply source of the polybutyl titanate, it may happen that this crosslinking agent must be worked into the formulation differently. For example, the crosslinking agent produced by Aldrich (Germany), after being dissolved in a small amount of ethanol, can simply be added all at once to the adhesive compound that contains the active substance. If the same procedure is followed with the crosslinking agent produced by Synetix (Vertec™, UK), brown particles form in the laminate after a few weeks. Therefore, this crosslinking agent must be predissolved in heptane, and then ethanol must be added to the mixture (mixing ratio 60:40), so that a 3% solution of the crosslinking agent is obtained. This solution is slowly added to the adhesive compound that contains the active substance, while the mixture is being vigorously stirred. Only then is a matrix obtained which is flawless even after storage.

It is recommended that the expert conduct preliminary tests to ensure that he proceeds carefully with the addition of the crosslinking agent, so that increased decomposition of fentanyl does not occur and especially that impurity D (European Pharmacopeia) does not form. This product already forms under conditions of stress storage of only one month at 40° C./75% relative humidity in an amount of about 1%, based on fentanyl. If the crosslinking agent is first homogenized in the adhesive compound in the absence of the active substance, and then the dissolved active substance is added, a laminate that is free of contaminant D should be obtained.

Another possibility for reducing the softening effect of fentanyl on the basic adhesive that is used is adjustment by admixture of a “harder” adhesive that is characterized by a content of vinyl acetate in the acrylate copolymer. This was successfully achieved by admixing an adhesive without functional groups, such as Durotak 87-4098. If Durotak types such as Durotak 87-2979 or 387-2287 or their successor types are used, then the ratio of 2-hydroxyethyl acrylate to vinyl acetate is no longer 1:0.4 to 1:5, but rather 1:5.2 or 1:6, and they thus no longer have the positive properties of high thermodynamic activity and the associated high in vitro release and in vitro skin permeation of the adhesive mixture in accordance with the invention, in which the ratio of hydroxyethyl acrylate to vinyl acetate is 1:0.4 to 1:5 in accordance with the invention. The following table provides an overview of the values obtained with the formulations that were tested:

| | | | Example 2 in | Example 3 in | Example 4 in |
|---|---|---|---|---|---|
| | | | Accordance | Accordance | Accordance |
| | Comparative | Comparative | with the | with the | with the |
| Parameter | Example 1 | Example 2 | Invention | Invention | Invention |
| Durotak 387- | 0% | 100% | 90% | 67% | 50% |
| 2510 | | | | | |
| Durotak 87- | 100% | 0% | 10% | 33% | 50% |
| 4098 | | | | | |
| ratio of 2- | 0% vinyl | 0% 2-hydroxy- | 1:5 | 1:2.2 | 1:0.6 |
| hydroxyethyl | acetate | ethyl acrylate | | | |
| acrylate: | | | | | |
| vinyl acetate | | | | | |
| adhesive | 3.9 | 9.1 | 8.3 | 7.1 | 6.7 |
| strength in | | | | | |
| vitro [N/25 | | | | | |
| mm] | | | | | |
| adhesive | adheres | Painful removal, | Good adhesive | Good adhesive | Good |
| properties in | relatively | including | property, slight | property, slight | adhesive |
| vivo | weakly | horny layer | adhesive borders | adhesive borders | property |
| aold flow | strongly | Not | barely | barely | not |
| | present | present | present | present | present |
| in vitro | -/29/-/ | -/62/-/ | -/69/-/ | -/57/-/ | not |
| release [%] | 46/103 | 88/104 | 89/95 | 75/84 | determined |
| of rated | | | | | |
| content after | | | | | |
| 1/2/3/4/6 h | | | | | |

It is apparent that the admixture of the small amount of 1/10 of the total amount of adhesive already reduces the in vitro adhesive properties, which also manifests itself in the in vivo wearing properties. The effect of the 10% addition on the in vitro release is still comparable to the release from 100% Durotak 387-2510; however, when the admixed amount of Durotak 87-4098 is increased to 30%, the release rate decreases. It was thus found, surprisingly, that the admixture of 10% Durotak 87-4098 results in optimum adhesive properties with unchanged release. In the formulations in accordance with the invention, actual application and placebo exhibit the same in vitro adhesive strengths.

The carrier of the matrix also plays an important role in the wearing properties. Since, in the strongest dosage with a delivery rate of 100 μg fentanyl per hour, the transdermal system already reaches a size of 40 cm², which is considerable, a certain degree of flexibility is an advantage with respect to wearing comfort.

Various transparent film materials were tested, which included, with respect to the chemistry of the material, PET (polyester), BOPP (biaxially oriented polypropylene), PE (polyethylene, polyolefins), PU (polyurethane), and PS (polystyrene copolymer). Another important consideration here was the extent to which fentanyl exhibited migration behavior relative to the materials. It was found that PU achieved no cohesion with the adhesive matrix and was therefore unsuitable. PE showed very pleasant wearing properties, but about 8-10% of the active substance migrated into this carrier film within less than one month at 40° C./75% relative humidity and was thus no longer available for transdermal absorption. Since fentanyl is very expensive as a raw material, one would not wish to remedy this problem by adding more fentanyl during production. This approach would also be unsuitable for the reason that the amount of fentanyl that migrates into the film changes over time. No migration was observed in PET (polyester), followed by BOPP, which was also preferred due to its somewhat greater flexibility.

A siliconized polyester film with which the expert is already familiar is used as the protective film, e.g., Hostaphan RN 100 by Mitsubishi, Germany, siliconizing easy/easy. The protective film should not be too thin (at least 36 μm layer thickness, and preferably 100 μm layer thickness), so that even the larger systems of 30 cm² or more can still be easily handled by the patient.

The dermal therapeutic systems are preferably constituted in such a way that they consist of a cover layer that is impermeable to the active substance, an adhesive layer that contains the active substance and adheres to the cover layer, and a removable protective layer.

This simplest form of a TDS can be produced in the manner well known to the expert by mixing a solution of the adhesive or adhesive mixture in a low-boiling solvent with the active substance, uniformly applying the mixture to a removable protective layer, quantitatively removing the solvent by heating, and covering the resulting product with a carrier. The applied adhesive layer containing the active substance has a thickness of 20 to 500 μm.

The following specific embodiments explain the invention in greater detail:

### EXAMPLE 1 IN ACCORDANCE WITH THE INVENTION

0.056 g of polybutyl titanate in the form of a 3% solution of heptane:ethyl alcohol 60:40 is slowly added with vigorous stirring to 23.44 g of a 42% (w/w) solution of an acrylate adhesive (Durotak 387-2510, National Starch & Chemical B.V., Zutphen, Netherlands), and the resulting mixture is homogenized. 1.1 g of fentanyl dissolved in 11.4 g of ethanol is added. The adhesive compound containing the active substance is homogenized by stirring for one hour and then spread with a doctor blade on a siliconized, 100-μm-thick polyester film (FL 2000, 100μ, 1-S, Loparex B.V., Apeldoorn, Netherlands) in a wet coating thickness of 310 μm. After drying (10 minutes at 70° C. and 5 minutes at 100° C.), the clear and homogeneous laminate is backed with a polyester film (Hostaphan RN15, Mitsubishi, Frankfurt, Germany). A patch with an area of 10 cm² contains 5.5 mg of fentanyl with a matrix weight of 55.0 g/m².

### EXAMPLE 2 IN ACCORDANCE WITH THE INVENTION

A solution of 0.33 g of fentanyl in 3.7 g of ethanol is added to a mixture of 6.29 g of a 42% (w/w) solution of the acrylate adhesive Durotak 387-2510 and 0.86 g of a 38.3% (w/w) solution of the acrylate adhesive Durotak 87-4089. The solution is homogenized by stirring for one hour and then spread with a doctor blade on a siliconized, 100-μm-thick polyester film (FL 2000, 100μ, 1-S, Loparex B.V., Apeldoorn, Netherlands) in a wet coating thickness of 400 μm. After drying (15 minutes at 70° C.), the slightly cloudy laminate is backed with a BOPP film (Trespaphan NAA, 40 μm, Trespaphan, Frankfurt, Germany). A patch with an area of 10 cm² contains 5.5 mg of fentanyl with a matrix weight of 55.0 g/m².

### EXAMPLE 3 IN ACCORDANCE WITH THE INVENTION

A solution of 0.33 g of fentanyl in 3.7 g of ethanol is added to a mixture of 4.71 g of a 42% (w/w) solution of the acrylate adhesive Durotak 387-2510 and 2.58 g of a 38.3% (w/w) solution of the acrylate adhesive Durotak 87-4089. The solution is homogenized by stirring for one hour and then spread with a doctor blade on a siliconized, 100-μm-thick polyester film (FL 2000, 100μ, 1-S, Loparex B.V., Apeldoorn, Netherlands) in a wet coating thickness of 400 μm. After drying (15 minutes at 70° C.), the slightly cloudy laminate is backed with a BOPP film (Trespaphan NAA, 40 μm, Trespaphan, Frankfurt, Germany). A patch with an area of 10 cm² contains 5.5 mg of fentanyl with a matrix weight of 55.0 g/m².

### EXAMPLE 4 IN ACCORDANCE WITH THE INVENTION

A solution of 0.33 g of fentanyl in 3.7 g of ethanol is added to a mixture of 3.54 g of a 42% (w/w) solution of the acrylate adhesive Durotak 387-2510 and 3.87 g of a 38.3% (w/w) solution of the acrylate adhesive Durotak 87-4089. The solution is homogenized by stirring for one hour and then spread with a doctor blade on a siliconized, 100-μm-thick polyester film (FL 2000, 100μ, 1-S, Loparex B.V., Apeldoorn, Netherlands) in a wet coating thickness of 400 μm. After drying (15 minutes at 70° C.), the slightly cloudy laminate is backed with a BOPP film (Trespaphan NAA, 40 μm, Trespaphan, Frankfurt, Germany). A patch with an area of 10 cm² contains 5.5 mg of fentanyl with a matrix weight of 55.0 g/m².

The following embodiment shows that a patch produced in accordance with the invention is bioequivalent to the originator product Durogesic in a crossover bioavailability study on six healthy subjects when the two patch products are worn for three days each.

The formulation was the same as Example 1 in accordance with the invention except that the backing consisted of a BOPP film (Trespaphan NAA, 40 μm, Trespaphan, Frankfurt, Germany) instead of a polyester film (Hostaphan RN15, Mitsubishi, Frankfurt, Germany). Each 10 cm² patch contained 5.5 mg of fentanyl with a matrix weight of 55.0 g/m². The comparative patch was the Durogesic™ 25 μg membrane patch. The pharmacokinetic results are compiled in the following table:

| | Example 1 in | |
|---|---|---|
| | Accordance with the | |
| | Invention | Durogesic ™ 25 μg |
| Name | Fentanyl TDS 25 | membrane patch |
| AUC (0-72 h) | 26.723 pg/mL*h | 24.911 pg/mL*h |
| C max | 496 pg/mL | 499 pg/mL |
| T max | 33 h (9 h) | 42 h |
| C peaks | 24-42 h | 30-71.8 h |
| absorption | somewhat faster | — |
| delivery rate | same | — |
| BA (AUC) | 107.2% (89-129.3%) | somewhat lower |
| BA (C max) | 99.5% (80.1-123.5%) | same |
| ANOVA CV (AUC) | 15.2% | — |
| n = 6 | | |
| ANOVA CV (C max) | 17.7% | — |
| n = 6 | | |

The skin tolerance and side effects were comparable for both products.

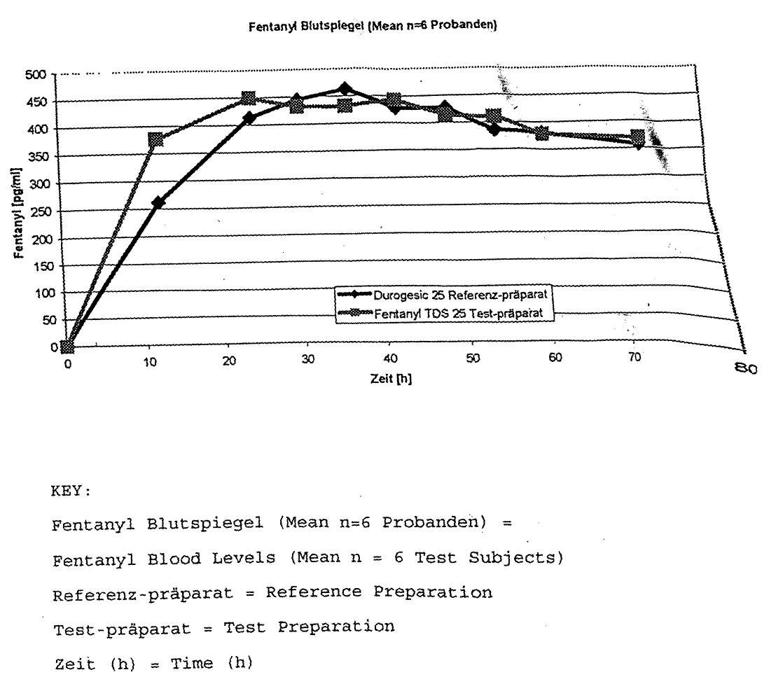

The graph in FIG. 1 shows the curves of the blood levels of the two products.

The drying conditions specified in the examples were the conditions used on the laboratory scale to produce the patches. The conditions used for production on a larger scale can differ from these laboratory conditions. For example, in an experimental-scale operation, the product may be conveyed at a rate of 2 m/minute through a tunnel drier with four drying zones with temperatures of 40° C., 60° C., 90° C. and 120° C. Production on a mass-production scale may involve different conditions, which are to be determined in scale-up tests.

1. Transdermal therapeutic system with a cover layer, an adhesive matrix that contains fentanyl as the active ingredient, and a removable protective layer, comprising an acrylate copolymer adhesive matrix that contains no penetration accelerators, such that the adhesive matrix is selected from the following group:
(a) basic acrylate copolymer, especially acrylate copolymer with hydroxyethyl acrylate units and with an organotitanium compound as a crosslinking agent; and
(b) basic acrylate copolymer with vinyl acetate units and free of crosslinking agents, especially acrylate copolymer with hydroxyethyl acrylate units and vinyl acetate units.

2. Transdermal therapeutic system in accordance with claim 1, comprising a fentanyl concentration of 0.1 to 30 wt. % and especially 5 to 18 wt. %, based on the weight of the adhesive matrix with the active substance.

3. Transdermal therapeutic system in accordance with claim 1, comprising a residual content of fentanyl solvent, especially ethyl alcohol, of less than 0.25 wt. %, based on the weight of the adhesive matrix with the active substance.

4. Transdermal therapeutic system in accordance with claim 1, comprising
(a) an acrylate copolymer comprising units that originate exclusively from 2-ethylhexyl acrylate, methacrylate, and 2-hydroxyethyl acrylate, or
(b) an acrylate copolymer comprising units that originate exclusively from 2-ethylhexyl acrylate, methacrylate, 2-hydroxyethyl acrylate, and vinyl acetate.

5. Transdermal therapeutic system in accordance with claim 1, comprising an acrylate copolymer as the adhesive matrix, which can be produced by drying at a temperature of about 70° C. or at a temperature above 70° C.

6. Transdermal therapeutic system in accordance with claim 1, characterized by the adhesive matrix comprising basic acrylate copolymer, especially acrylate copolymer with hydroxyethyl acrylate units and with an organotitanium compound as a crosslinking agent, which can be produced by crosslinking of the hydroxyl groups of the acrylate copolymer and subsequent addition of the active substance.

7. Transdermal therapeutic system in accordance with claim 1, characterized by the adhesive matrix comprising basic acrylate copolymer with vinyl acetate units and free of crosslinking agents, especially acrylate copolymer with hydroxyethyl acrylate units and vinyl acetate units, which can be produced from 2-ethylhexyl acrylate, methacrylate, 2-hydroxyethyl acrylate, and vinyl acetate in a ratio of 2-hydroxyethyl acrylate:vinyl acetate of 1:0.3 to 1:5, preferably 1:0.4 to 1:5, more preferably 1:0.6 to 1:5, and most preferably 1:2.2 to 1:5, in each case on the basis of the number of moles or on the basis of weight.

8. Transdermal therapeutic system in accordance with claim 7, characterized by the adhesive matrix comprising basic acrylate copolymer with vinyl acetate units and free of crosslinking agents, especially acrylate copolymer with hydroxyethyl acrylate units and vinyl acetate units, which can be produced from 2-ethylhexyl acrylate, methacrylate, 2-hydroxyethyl acrylate, and vinyl acetate in a ratio of 2-hydroxyethyl acrylate:vinyl acetate of 1:1.5 to 1:3.0, and especially about 1:2.2, in each case on the basis of the number of moles or on the basis of weight.

9. Transdermal therapeutic system in accordance with claim 1, characterized by the adhesive matrix comprising basic acrylate copolymer, especially acrylate copolymer with hydroxyethyl acrylate units and with an organotitanium compound as a crosslinking agent with polybutyl titanate as the crosslinking agent.

10. Transdermal therapeutic system in accordance with claim 9, comprising a concentration of polybutyl titanate in an amount of 0.1 to 1%, and preferably 0.4 to 0.6%, calculated on the basis of the number of moles.

11. Transdermal therapeutic system in accordance with claim 1, comprising a layer thickness of the adhesive matrix of 20 to 500 μm.

12. Transdermal therapeutic system in accordance with claim 1, comprising a cover layer based on polypropylene and especially by a biaxially oriented, longitudinally and transversely aligned polypropylene film.

13. Transdermal therapeutic system in accordance with claim 1, comprising a cover layer based on polyester and especially by a polyester fabric.

14. Transdermal therapeutic system in accordance with claim 1, wherein the cover layer is designed as a matrix carrier.
